(19) United States
(12) Patent Application Publication
(10) Pub. No.: US 2025/0120814 A1
(43) Pub. Date: Apr. 17, 2025

(54) IMPLANTABLE UROLOGICAL DEVICE WITH POWER AND COMMUNICATION INTERFACE

(71) Applicant: Boston Scientific Scimed, Inc., Maple Grove, MN (US)

(72) Inventors: Jean M. Bobgan, Maple Grove, MN (US); Joey Chen, Stevenson Ranch, CA (US); Keith R. Maile, New Brighton, MN (US); Daniel J. Landherr, Wyoming, MN (US)

(21) Appl. No.: 18/908,171

(22) Filed: Oct. 7, 2024

Related U.S. Application Data
(60) Provisional application No. 63/589,908, filed on Oct. 12, 2023.

(51) Int. Cl. A61F 2/26

(52) U.S. Cl. CPC A61F 2/26; A61F 2220/0025; A61F 2250/0002; A61F 2250/0069; A61F 2250/0078

(57) ABSTRACT

An implantable urological device is disclosed. The implantable urological device includes a housing forming a first internal compartment, an electronic component disposed within the first internal compartment, and a header coupled to the housing. The header forms a second internal compartment. A secondary conductor is disposed within the second internal compartment and electrically coupled to the electronic component. The secondary conductor receives a wireless power signal. An antenna is disposed within the second internal compartment and electrically coupled to the electronic component. The antenna receives a wireless communication signal.

### CROSS-REFERENCE TO RELATED APPLICATION

This application claims priority to U.S. Provisional Patent Application No. 63/589,908, filed on Oct. 12, 2023, entitled “IMPLANTABLE UROLOGICAL DEVICE WITH POWER AND COMMUNICATION INTERFACE”, the disclosure of which is incorporated by reference herein in its entirety.

### TECHNICAL FIELD

The present disclosure relates generally to medical systems and implantable medical devices. More specifically, the present disclosure relates to implantable medical devices that recharge and communicate in medical systems.

### BACKGROUND

Implantable medical devices include electrical or electromechanical medical devices that are implanted within a patient and perform a task such as to monitor a parameter of the patient or to deliver a therapy to the patient via electrical energy. For example, the implantable medical device can be an implantable urological device implanted into the patient to treat a condition such as erectile disfunction, penile deformity, or incontinence as an implantable fluid operated inflatable device. Some implantable medical devices are designed to receive communication signals from external devices of a medical system. Many implantable medical devices are designed to receive power directly from an energy storage system such as a battery or capacitor located with the implantable medical device, but the energy storage system can be depleted of energy long before the end of the useful life of the implantable medical device. In some examples, the implantable medical device includes a rechargeable energy storage system such as a rechargeable battery to extend the life of the implantable medical device. A wireless charger may be applied to recharge a depleted battery in the implanted medical device.

### SUMMARY

In Example 1, an implantable urological device comprising: a housing forming a first internal compartment; an electronic component disposed within the first internal compartment; a header coupled to the housing, the header forming a second internal compartment; a secondary conductor disposed within the second internal compartment and electrically coupled to the electronic component, the secondary conductor configured to receive a wireless power signal; and an antenna disposed within the second internal compartment and electrically coupled to the electronic component, the antenna configured to a receive a wireless communication signal.

In Example 2, the implantable urological device of Example 1, wherein the electronic component includes a treatment system and a communication system.

In Example 3, the implantable urological device of any of Example 1 and 2, further comprising a rechargeable power source coupled to the electronic component and disposed within the first internal compartment, wherein the electronic component is configured to charge the rechargeable power source from the power signal received from the secondary conductor.

In Example 4, the implantable urological device of any of Examples 1-3, wherein the implantable medical device is included in an implantable penile prosthesis.

In Example 5, the implantable urological device of Example 4, further including a reservoir having a fluid, and the implantable medical device is in fluid communication with the reservoir.

In Example 6, the implantable urological device of Example 5, further including device includes a plurality of inflatable cylinders to receive the fluid, and the implantable medical device is in fluid communication with the plurality of inflatable cylinders, and wherein the implantable medical device is configured to pump the fluid from the reservoir to the plurality of inflatable cylinders.

In Example 7, the implantable urological device of any of Examples 1-6, wherein the implantable urological device is included in a medical system further comprising a remote charger and a remote programmer wherein the remote charger is wirelessly coupleable to the secondary coil to provide the transcutaneous power transfer, and wherein the programmer is in radiofrequency communication with the electronic component via the antenna.

In Example 8, the implantable urological device of Example 7, wherein the programmer includes a software application operating on a mobile computing device to activate the implantable urological device.

In Example 9, the implantable urological device of any of Example 1-8, wherein the first internal compartment is hermetically sealed from the second internal compartment, and the secondary conductor and the antenna are electrically coupled to the electronic component via a hermetic feedthrough system.

In Example 10, the implantable urological device of Example 9, wherein the hermetic feedthrough system is a common feedthrough electrically and mechanically coupling the secondary conductor and the antenna to the electronic component via a switch configured to select one of the power signal and the communication signal.

In Example 11, the implantable urological device of Example 9, wherein the hermetic feedthrough system includes a secondary coil feedthrough electrically and mechanically coupling the secondary conductor to a recharge system of the electronic component and wherein the hermetic feedthrough system includes an antenna feedthrough electrically and mechanically coupling the antenna to a communication system of the electronic component.

In Example 12, the implantable urological device of any of Examples 9-11, wherein the antenna is a monopole stub mechanically coupled to the secondary conductor.

In Example 13, the implantable medical device of any of Examples 9-12, wherein the housing is electrically conductive, and the secondary conductor includes a first end electrically coupled to the feedthrough system and a second end electrically coupled to the housing.

In Example 14, the implantable urological device of any of Examples 9-12, wherein the housing is electrically conductive, and the secondary conductor is electrically insulated from the housing.

In Example 15, the implantable urological device of any of Examples 1-8, wherein the first and second internal compartments are hermetically sealed within the housing and header, and at least one of the of the secondary conductor and the antenna include copper.

In Example 16, an implantable urological device, comprising: a housing forming a first internal compartment; an electronic component and a rechargeable power source disposed within the first internal compartment, the electronic component including a treatment system, a communication system, and a recharge system, the recharge system coupled to the rechargeable power source; a secondary conductor disposed within the second internal compartment and electrically coupled to the electronic component, the secondary conductor configured to receive a wireless power signal, wherein the recharge system is configured to charge the rechargeable power source from the power signal received from the secondary conductor; and an antenna disposed within the second internal compartment and electrically coupled to the electronic component, the antenna configured to a receive a wireless communication signal, wherein the communication signal is provided to the communication system.

In Example 17, the implantable urological device of Example 16, wherein the power source is a rechargeable power source including a rechargeable battery.

In Example 18, the implantable urological device of Example 17, wherein the implantable urological device is an inflatable penile prosthesis.

In Example 19, the implantable urological device of Example 18, and further comprising a fluidics circuit disposed within the housing and electrically coupled to the treatment system.

In Example 20, the implantable urological device of Example 19 wherein housing includes a third internal compartment, the fluidics circuit disposed within the third internal compartment, and the third internal compartment hermetically sealed from the first and second internal compartments.

In Example 21, the implantable urological device of Example 20, wherein the fluidics circuit includes a pump assembly.

In Example 22, the implantable urological device of Example 20, wherein the fluidics circuit includes a manifold hermetically sealed to the housing to form the third internal compartment.

In Example 23, the implantable urological device of Example 16, wherein the first internal compartment is hermetically sealed from the second internal compartment, and the secondary conductor and the antenna are electrically coupled to the electronic component via a hermetic feedthrough system.

In Example 24, the implantable urological device of Example 23, wherein the hermetic feedthrough system is a common feedthrough electrically and mechanically coupling the secondary conductor and the antenna to the electronic component via a switch configured to select one of the power signal and the communication signal.

In Example 25, the implantable urological device of Example 23, wherein the hermetic feedthrough system includes a secondary coil feedthrough electrically and mechanically coupling the secondary conductor to a recharge system of the electronic component and wherein the hermetic feedthrough system includes an antenna feedthrough electrically and mechanically coupling the antenna to a communication system of the electronic component.

In Example 26, the implantable urological device of Example 23, wherein the antenna is a monopole stub mechanically coupled to the secondary conductor.

In Example 27, the implantable urological device of Example 23, wherein the housing is electrically conductive, and the secondary conductor includes a first end electrically coupled to the feedthrough system and a second end electrically coupled to the housing.

In Example 28, the implantable urological device of Example 23, wherein the housing is electrically conductive, and the secondary conductor is electrically insulated from the housing.

In Example 29, the implantable urological device of Example 16, wherein the first and second internal compartments are hermetically sealed within the housing and header, and at least one of the of the secondary conductor and the antenna include copper.

In Example 30, an implantable urological device, comprising: a housing forming a first internal compartment; an electronic component disposed within the first internal compartment; a header coupled to the housing, the header forming a second internal compartment; a secondary conductor disposed within the second internal compartment and electrically coupled to the electronic component, the secondary conductor configured to receive a wireless power signal; and an antenna disposed within the second internal compartment and electrically coupled to the electronic component, the antenna configured to a receive a wireless communication signal.

In Example 31, the implantable urological device of Example 30, wherein the implantable urological device is included in a medical system further comprising a remote charger and a remote programmer wherein the remote charger is wirelessly coupleable to the secondary coil to provide the transcutaneous power transfer, and wherein the programmer is in radiofrequency communication with the electronic component via the antenna.

In Example 32, the implantable urological device of Example 31, wherein the programmer includes a software application operating on a mobile computing device to activate the implantable medical device.

In Example 33, an implantable urological device, comprising: a reservoir configured to receive a fluid; and an inflatable member in fluid communication with the reservoir. The implantable medical actuation device, comprising: a housing forming a first internal compartment; an electronic component and a rechargeable power source disposed within the first internal compartment, the electronic component including a treatment system, a communication system, and a recharge system, the recharge system coupled to the rechargeable power source; a header coupled to the housing, the header forming a second internal compartment; a secondary conductor disposed within the second internal compartment and electrically coupled to the electronic component, the secondary conductor configured to receive a wireless power signal, wherein the recharge system is configured to charge the rechargeable power source from the power signal received from the secondary conductor; and an antenna disposed within the second internal compartment and electrically coupled to the electronic component, the antenna configured to a receive a wireless communication signal, wherein the communication signal is provided to the communication system.

In Example 34, the implantable urological device of Example 33, wherein the inflatable member includes a plurality of inflatable cylinders in fluid communication with the medical actuation device and the reservoir, the plurality of inflatable cylinders configured to be disposed within a corpora cavernosa of a penis.

In Example 35, the implantable urological device of Example 33, wherein the medical instrument is configured to be disposed within a retropubic space.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example medical system including an example implantable medical device, an example external charger, and an example external programmer of the present disclosure.

FIG. 2 is a schematic diagram illustrating an example implantable urologic device including features of the example implantable medical device of FIG. 1.

FIG. 3 is an exploded view of an example feature of the example implantable urologic device of FIG. 2.

FIG. 4A is a perspective view of an embodiment of an example feature of the example implantable urologic device of FIG. 2.

FIG. 4B is a perspective view of an embodiment of an example feature of the example implantable urologic device of FIG. 2.

FIG. 4C is a perspective view of an embodiment of an example feature of the example implantable urologic device of FIG. 2.

FIG. 4D is a perspective view of an embodiment of an example feature of the example implantable urologic device of FIG. 2.

FIG. 4E is a perspective view of an embodiment of an example feature of the example implantable urologic device of FIG. 2.

FIG. 5A is a perspective view of an embodiment of an example feature of the example implantable urologic device of FIG. 2.

FIG. 5B is a perspective view of an embodiment of an example feature of the example implantable urologic device of FIG. 2.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

Wireless power transfer or transmission is used to deliver power from a power source without a mechanical connection to electronic devices. Wireless power transfer systems are used in a variety of applications, such as, for recharging batteries in mobile computing devices such as smart phones or wearable devices. Wireless power transfer systems are also used to transmit power transcutaneously, or through the skin, to medical devices implanted in a patient either to directly power the implanted medical device or to recharge an energy storage system of the implanted medical device. Examples of wireless power transfer systems include inductive charging and radiofrequency (RF) charging.

In some examples, transcutaneous charging is performed via inductive power transfer or transmission, which is presented here for illustration. The energy storage system of the implantable medical device can be recharged with an external charger configured to provide inductive power transfer. Inductive power transfer can be performed with an inductive coupling between conductors, which may be formed as coils of wire, such as a primary conductor, which can include a primary coil, in the charger and a secondary conductor, such as for example a secondary coil, in the implantable medical device. Power is transferred from the primary conductor to the secondary conductor with a magnetic field. An alternating current through the primary conductor creates an oscillating magnetic field. The magnetic field passes through the secondary conductor, and the magnetic field induces an alternating electromotive force, or EMF, such as voltage, which creates an alternating current in the secondary conductor. The induced alternating current may either directly drive a load in the implantable medical device or be rectified to direct current by a rectifier in the implantable medical device, which drives the load. Resonant inductive coupling is a type of inductive coupling in which power is transferred by magnetic fields between two resonant circuits, one in the charger and one in the implantable medical device. Each resonant circuit can include a coil of wire connected to a capacitor, or a self-resonant coil or other resonator with internal capacitance. Resonant circuits, or tank circuits, are tuned to resonate at generally the same resonant frequency. The resonance between the conductors can greatly increase coupling and power transfer between the charger and the implantable medical device. In this example, the external charger does not mechanically connect with the implantable medical device, and the external charger can be used to charge the implantable medical device from a relatively short distance away.

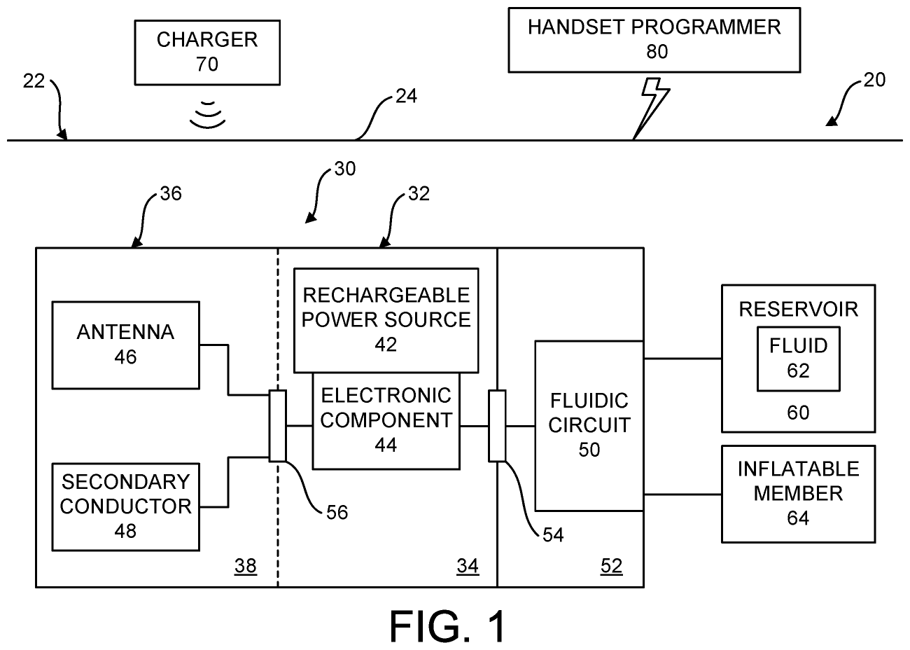

FIG. 1 illustrates an embodiment of a medical system 20. The medical system 20 includes an implantable medical device 30, which can be fully implanted within a patient 22. The implantable medical device 30 includes a housing 32 forming first internal compartment 34 within the implantable medical device 30. The implantable medical device 30 includes a header 36 coupled to the housing 32. The header forms a second internal compartment 38 within the implantable medical device 30. The implantable medical device 30 can include an energy storage system, such as a rechargeable power source 42 and an electronic component 44 within the first internal compartment 34. An antenna 46 is disposed within the second internal compartment 38 and is electrically coupled to the electronic component 44. The antenna 46 is configured to a receive a wireless communication signal, and, in some embodiments, the antenna is configured to transmit a wireless communication signal as well. A secondary conductor 48 is disposed within the second internal compartment 38 and is electrically coupled to the electronic component 44. The secondary conductor 48 is configured to receive a wireless power signal. In some embodiments, the wireless power signal is provided to the electronic component 44 to charge the rechargeable power source 42. In some embodiments, the housing 32 includes a fluidics circuit 50 disposed within a third internal compartment 52. The third internal compartment 52 is hermetically isolated from the first internal compartment 34, such as via a hermetic seal or frame. The electronic component is electrically coupled to the fluidics circuit 50 such as via a hermetic interface 54 to operate the fluidics circuit 50. In some embodiments, the first internal compartment 34 is hermetically isolated from the second internal compartment 38, and the antenna 46 and secondary conductor 48 are electrically coupled to the electronic component 44 via a hermetic feedthrough connection 56.

The illustrated implantable medical device 30 is coupled to a reservoir 60 filled with a fluid 62, such as a sterile saline solution, in fluid communication with the fluidics circuit 50. The implantable medical device 30 is coupled to an inflatable member 64 in fluid communication with the fluidics circuit 50 and the reservoir 60 to receive the fluid 62. The electronic component 44 provides for the monitoring and control of various operations of the fluidics circuit 50. The fluidics circuit 50 can provide for the transfer of fluid 62 between the reservoir 60 and the inflatable member 62. In some embodiments, the electronic components 44 can include a recharge system, a communication system, and a control system. In some embodiments, the fluidics circuit 50 can include a manifold, and fluidics components such as a pump assembly, a valve assembly, and a pressure sensor.

The implantable medical system 20 also includes a charger 70, which can also be referred to as a wireless recharger, external to outside of the patient 22, or across transcutaneous boundary 24 such as the surface of the patient's skin proximate the implantable medical device 30 to provide power signals and, in some examples, telemetry to the medical device 30. In some embodiments, transcutaneous charging is performed via radiofrequency power transfer or transmission or inductive power transfer or transmission. The charger 70 does not mechanically connect with the implantable medical device 30, and the charger 70 can be used to charge the implantable medical device 30 from a relatively short distance away. For example, the charger 70 is placed against the patient 22 and proximate the implantable medical device 30 to inductively transfer energy and to replenish the rechargeable power source 42.

In an embodiment of the charger 70, inductive power transfer can be performed with an inductive coupling between conductors, which may be formed as coils of wire, such as a primary coil in the charger 70 and the secondary conductor 48 in the implantable medical device 30. Power is transferred from the charger 70 to the secondary conductor 48 with a magnetic field. An alternating current through the primary coil creates an oscillating magnetic field. The magnetic field passes through the secondary conductor 48, and the magnetic field induces an alternating electromotive force, or EMF, such as voltage, which creates an alternating current in the secondary conductor 48. The induced alternating current may either directly drive a load in the implantable medical device 30 or be rectified to direct current by a rectifier in the implantable medical device 30, such as part of the electronic component 44, which drives the load. In some embodiments, the induced current is applied to replenish the rechargeable power source 42 via the electronic circuit 44, and in some embodiments the induced current is used to drive the fluidics circuit 50 via the electronic component 44. Resonant inductive coupling is a type of inductive coupling in which power is transferred by magnetic fields between two resonant circuits, one in the charger 70 and one in the implantable medical device 30. Each resonant circuit can include a coil of wire connected to a capacitor, or a self-resonant coil or other resonator with internal capacitance. Resonant circuits, or tank circuits, are tuned to resonate at generally the same resonant frequency. The resonance between the conductors can greatly increase coupling and power transfer between the charger 70 and the implantable medical device 30.

In the example, the charger 70 delivers magnetic energy to a corresponding implantable device 30 at the preselected frequency with a resonant inductor-capacitor (LC) tank circuit to generate an H-field. The tank circuit includes a recharge coil in series with a recharge capacitor. Various configurations of the charger 70 can share a common coil design, and the preselected recharge frequency is determined via a selected tank capacitance of the recharge capacitor. The tank circuit can oscillate at a resonant frequency. A phase locked loop in the tank circuit is created via pulsing an applied tank voltage in phase with a tank current. During resonance, the tank current is approximately or generally sinusoidal over time. The tank circuit can achieve maximum tank power when a tank voltage pulse is aligned in time with the tank current. Recharge power can be adjusted by altering the magnitude and duty of the tank voltage pulse input to the tank circuit.

The charger 70 is available in different configurations depending on recharge frequencies and communication schemes for use with the implantable medical device 30. For example, a first configuration of the charger 70 may support a bidirectional inductive telemetry communication scheme and a first recharge frequency, a second configuration of the charger 70 may support a radiofrequency telemetry and downlink inductive telemetry communication schemes and a second recharge frequency, and a third configuration of the charger may support the bidirectional inductive telemetry communication scheme and a third recharge frequency. Inductive charging is presented in this disclosure for illustration, and the charger 70 may use other forms of wireless charging to replenish the rechargeable power source.

System 20 can also include a handset programmer 80 configured to wirelessly interface and communicate with the implantable medical device 30 or with the charger 70, also external to or outside of the patient 22. In one example, the handset programmer 80 can be implemented as a general-purpose computing device or mobile computing device that hosts a software application. For instance, the handset programmer 80 can include a set of controls to transcutaneously communicate with or operate the implantable medical device 30 via the communication system or to communicate with or operate the charger 70. The medical device 30 incudes the antenna 46 to receive and transmit communication signals with the handset programmer 80. For example, the handset programmer 80 and medical device 30 can communicate via radiofrequency signals in the 2.400-2.4835 GHz range as in Bluetooth Low Energy communication in, for example, a wireless personal network. Additionally, or alternatively, the handset programmer 80 or charger 70 may apply other forms of wireless communication, which can include other forms of radiofrequency communication or inductive communication. The handset programmer 80 may be configured with a user interface, such as a graphical user interface, to receive commands such as via soft buttons to wirelessly operate the medical device 30, receive feedback or parameters form the medical device 30, update a control system on the medical device, and interface with a computer network to provide telemetry or to allow control of the medical device 30 by a remote, computer network-connected system. For example, a user can apply the handset programmer 80 to actuate the medical device 30 such that the fluid 62 is pumped from the reservoir 60 into the inflatable member 64 or to release fluid 62 from the inflatable member 64 to return to the reservoir 60.

Systems of the present disclosure can optionally include additional components. The system 20 can include a charging dock, which can be plugged into a wall outlet and configured to charge an internal battery of the charger 70. The charger 70 can also be used in conjunction with a fixation product of system 20 to keep the charger 70 in position proximate the implantable medical device 30 during a recharge session. The fixation product can include a fixation belt to be worn around a portion of the patient 22 such as the belt line for an implantable medical device 30 in the abdomen, back, buttocks, or flank of the patient 22, or a fixation drape to be worn around the neck with a counterweight to balance the charger 70 for an implantable medical device 30 in the pectoral region of the patient 22. The fixation product receives the charger 70 to hold the charger 70 in place with respect to the fixation product so that the charger 70, in one example, does not rotate and generally does not move with respect to the implantable medical device 30 during the recharge session and to secure the charger 70 so as not to fall out unless purposefully removed from the fixation product.

The implantable medical device 30 can be implanted subcutaneously in an implantation locations or pockets within a patient 22. In some examples, another component of the implantable medical device 30 can occupy the same or an additional location within the patient 22. The implantable medical device 30 may be configured to deliver therapy to a patient, monitor parameters within a patient, receive and deliver signals with a patient, such as at regular intervals, continuously, or in response to a detected event such as an event detected by sensors, received from another implantable device (not shown), or received from components of the implantable medical system 20 such as from charger 70 or from handset programmer 80. The implantable medical device 30 can be configured to detect a variety of physiological signals that may be used in connection with various diagnostics, therapeutic, and other monitoring implementations. The implantable medical device 30 can be used in urological, neurological, cardiac, and other applicable fields that apply implantable medical devices with power systems or for receiving and transmitting signals.

In certain embodiments, the implantable medical device 32 is configured as a urological therapy device to deliver selective stimulation to, for example, the sacral nerves for treatment of urological disorders such as bladder control or erectile disorders. In other examples, the implantable medical device is configured as a gastrointestinal device to treat, for instance, gastroesophageal reflux disease (GERD). In other examples, the implantable medical device 32 may be configured as a neurostimulation therapy device for pain management and the like. In still other examples, the implantable medical device 32 may be a cardiac rhythm management device for sensing and stimulating cardiac tissue for treatment of cardiac arrhythmias such as bradycardia, tachycardia and for cardiac resynchronization therapy. In still other examples, the implantable medical device 32 may be configured as a monitoring device only, with no therapeutic functionality, to monitor physiological parameters of a patient. In short, the present disclosure is not limited to any clinical application, and any implantable device that requires power to operate as intended.

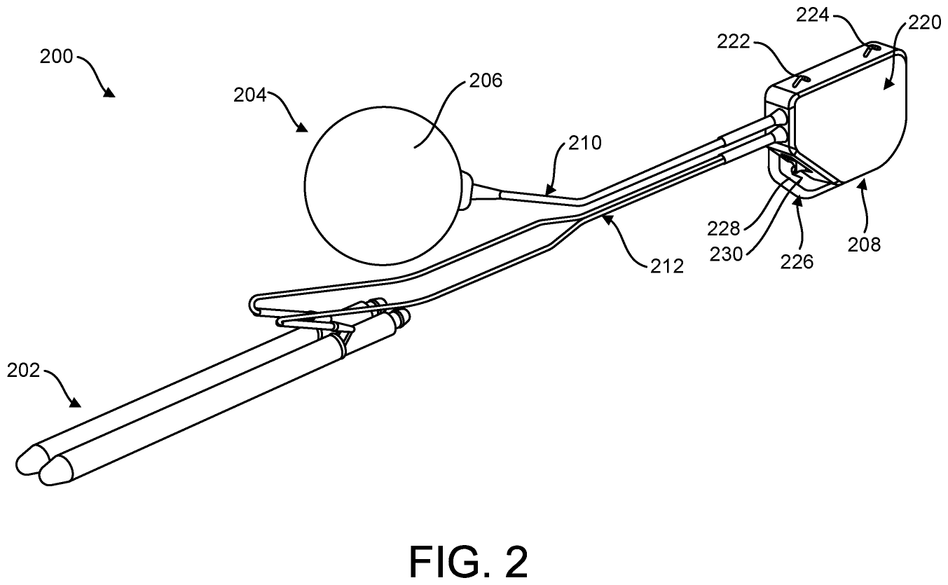

FIG. 2 illustrates an example implantable medical device 200 such as an implantable urologic device in connection with an inflatable penile prosthesis, which may correspond with implantable medical device 30. In the example implantable medical device 200 includes an inflatable member, such as a pair of inflatable cylinders 202, a reservoir 204 that may be filled with a fluid, such as a sterile saline solution 206, and an electromechanical actuation device 208 in a closed system. The reservoir 204 is fluidically coupled to the actuation device 208 via tubing 210, and the actuation device 208 is fluidically coupled to the cylinders 202 via tubing 212. For example, tubing 210, 212 can be kink-resistant tubing made from a silicone elastomer.

The actuation device 208 includes a hermetically sealed housing 220 formed of a biocompatible material such as titanium or steel. In one example, the housing 220 is formed via a plurality of walls welded together. The actuation device 208 includes an internal fluidics circuit to fluidically couple the reservoir 204 to the cylinders 202 via tubing 210, 212. Internal electronic component, powered by a rechargeable power source, can provide for the monitoring and control of various operations of the fluidics circuit such as the transfer of fluid 206 between the reservoir 204 and the cylinders 202. In one example, the electromechanical actuation device 208 can correspond with implantable medical device 30, the reservoir 204 and fluid 206 can correspond with reservoir 60 and fluid 62, respectively, and cylinders 202 can correspond with inflatable member 64 of FIG. 1.

In one embodiment, the housing 220 is configured to carry the rechargeable energy storage system, the electronics components, and the fluidics circuit. In some embodiments, the housing includes a plurality of compartments that can be separated by a frame and hermetically sealed from one another. The electronic component can be implemented by various components including resistors, capacitors, transistors, and integrated circuits disposed on one or more printed circuit boards within a first internal compartment. The fluidic circuit can be implemented via titanium manifolds and electromagnetic pumps or piezoelectric pumps in another internal compartment. In one example, the housing 220 can including an attachment device, such as loops 222, 224 formed a wall to receive sutures that can be applied to anchor the actuation device 208 to a structure within the patient.

The actuation device 208 includes a header 226 to form a second internal compartment that includes power and communication interface structures such as a secondary conductor 228 and an antenna 230. The header 226 is configured to allow the transmission of power and communication signals between the secondary conductor 228 and the charger 70 and between the antenna 230 and a handset programmer 80 or charger 70. The secondary conductor 228 and antenna 230 are electrically coupled to the electronic components within the first partition. In embodiments in which the first internal compartment is hermetically sealed from the second internal compartment, the secondary conductor 228 and the antenna 230 are coupled to the electronic component via a hermetic feedthrough connection.

The cylinders 202 are typically implanted in the corpora cavernosa of the penis, and the reservoir 204 are often implanted in the retropubic space, or Retzius space, of the patient or between the transverse muscle and the rectus muscle. The electromechanical actuation device 208 can be implanted in the abdomen at a selected location determined by a clinician. The sterile saline solution 206 can be pumped from the reservoir 204 into the chambers of the cylinders 202 via tubing 210, 212 with the actuation device 208. For example, the actuation device 208 may include electromechanical pumps and valves within the fluid circuit. The cylinders 202 replace non-functioning or limited-functioning erectile tissue in the penis, and the cylinders 202 inflated with the saline solution 206 produce an erection. After sexual intercourse, the actuation device 208 releases the saline solution 206 in the cylinders 202, and the fluid is returned to the reservoir 204 via the tubing and fluidic circuit within the actuation device 208.

In another example of implantable medical device incorporating the features of an electromechanical actuation device incorporating features of medical device 30, the inflatable member 64 can correspond with an inflatable cuff, which may be implemented as an artificial urinary sphincter. In some embodiments, the inflatable cuff is disposed about a urethra at a location proximate the bladder. The actuation device is applied to pump fluid from a reservoir to expand the cuff and to close the urethra. The cuff is deflated to allow a patient to void the bladder. The disclosure describes features of the medical device 30 with reference to a penile prothesis, such as medical device 200, for illustration, and the features of the medical device 30 can apply to other implementations and components.

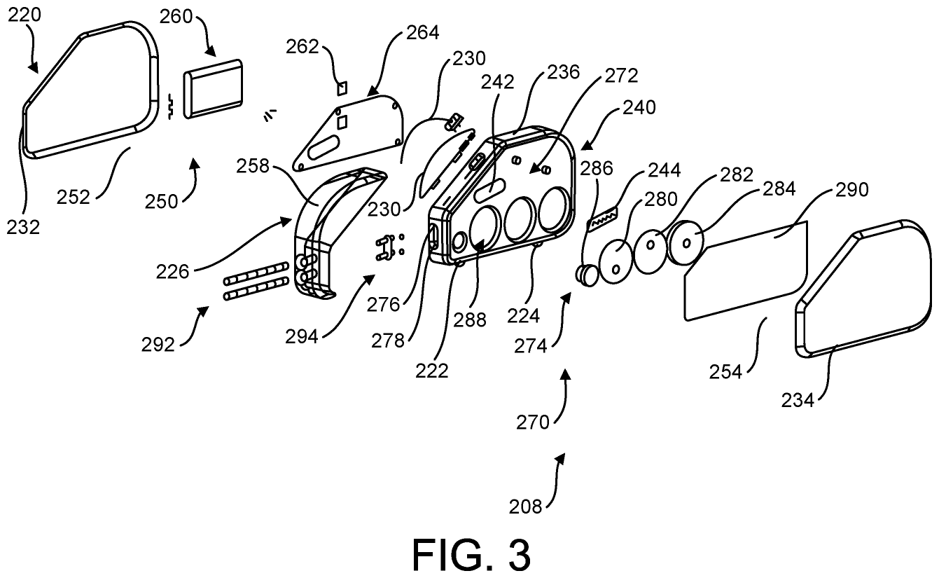

FIG. 3 illustrates an exploded view of the example actuation device 208 of FIG. 2 in which like parts are labeled with like reference numerals. The housing 220 includes a first sidewall 232, a second, opposite side wall 234, a peripheral wall 236, and a frame 240. In the example actuation device 208, the first sidewall 232, second sidewall 234, and peripheral wall 236 are hermetically sealed together. The frame 240 is disposed within the housing 220 to form a first internal compartment 252 and a third internal compartment 254 in such a manner that the first internal compartment 252 is hermetically sealed from the third internal compartment 254. In an example, the frame 240 can be integrally formed with the peripheral wall 236 or the first or second sidewall 232, 234. In another example, the frame 240 is welded to the peripheral wall 236 or welded to the first or second sidewall 232, 234. In the illustrated example, the first side wall 232, peripheral wall 236, and frame 240 form the first internal compartment 252. The second side wall 234, peripheral wall 236, and frame 240 form the internal compartment 254.

The actuation device 208 can include a header 226 attached to the housing 220 to form a second internal compartment 258 between an inner surface of the header 226 and an outer surface of the housing 220 that includes power and communication interface structures such as the secondary conductor 228 and the antenna 230. In one embodiment, the second internal compartment 258 is external to the first internal compartment 252 of the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may be comprised of an over-molded polymer affixed to the housing 220 and including the secondary coil 228 and antenna within the internal region 258. In this example, the secondary conductor 228 and antenna 230 are constructed from a biocompatible material.

The actuation device 208 includes an energy storage system, such as a rechargeable power source 260 or a rechargeable battery, and electronic component 262 within the first internal compartment 252. The electronic component 262 can be disposed on a circuit board 264, such as a plurality of circuit boards, within the first internal compartment 252. The rechargeable battery 260 can assume various forms appropriate to provide power for generating desired electrical signals and to store power provided from the electronic components 262. For example, the battery 260 can incorporate lithium-ion (Li+) chemistry, i.e., a lithium-ion battery to operate the electronic component 262. In one example, the electronics component 262 can be implemented by various components including resistors, capacitors, transistors, and integrated circuits disposed on the circuit board 264. The secondary coil 228 and antenna 230 are electrically coupled to the electronic components 262 within the first partition 252, such as via a hermetic feedthrough connection.

The electronic components 262 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as Bluetooth Low Energy, which is compatible with several operating systems that can be applied in mobile devices configured as handset programmers. Communication system can include an integrated circuit to implement an applied communication technology. In some examples, the communication system can be used to transmit communication signals to other devices, such as a charger or the handheld programmer, and the communication system can be implemented to generate communication signals and provide the communication signals to the antenna 230 for transmission. In some examples, the communication system can be configured to receive and transmit radiofrequency signals via the antenna 230. The controller can include a microcontroller to operate the recharge system and to receive and operate in response to communication signals or generate communication signals from communication system.

The actuation device 208 also includes a fluidics circuit 270 within the third internal compartment 254 and opposite the frame 240 from the battery 260 and electronic component 262. In the example, the frame 240 can include an opening 242 that includes a hermetic interface 244, such as a feedthrough hermetically affixed to the frame. The electronic components 262 are operably coupled to the fluidics circuit 270 across the frame 240 via the hermetic interface 244. For example, the controller of the electronic component 262, powered by the battery 260, can cause the operation of the fluidics circuit 270 such as to control and monitor the fluidics circuit 270. The fluidics circuit 270 includes a manifold 272 and fluid components 274 operably coupled to the manifold 272. In the illustrated example, the manifold 272 is a structure integrated into the frame 240 such that the manifold 272 and frame 240 together form the hermetic barrier between the first internal compartment 252 and the second internal compartment 254. For instance, the battery 260, the circuit board 264, or electronic components 262 can be coupled to a first major surface of the manifold 272 in the first internal compartment 252, and the fluidics components 274 are operably coupled to a second, and opposite major surface of the manifold 272 in the second internal compartment 254.

The fluidics circuit 270 provides for the transfer of the fluid 206 between the reservoir 204 and the inflatable member such as cylinders 202. The manifold 272, which can be a hermetic manifold, segments and contains the fluid from the first internal compartment 252 to reduce the chance of fluid exchange and directs the fluid from a first port 276 to a second port 278 via internal fluid passageways or channels. In the illustrated example, the fluidics components 274 include a plurality of fluid pumps, such as pumps 280, 282, a valve 284 mounted into the manifold 272 in fluidic communication with a manifold passageway to transfer fluid from the first port 276 to the second port 278. In an example fluid architecture, the pumps and valve are in fluid communication with a single fluid passageway between ports 276, 278. The fluidics components 274 also includes a pressure sensor 286 operably coupled to the manifold 272 and in fluidic communication with the passageway to detect a pressure of the fluid within the manifold 272. As indicated, the fluidics components 274 are included in a planar configuration on the manifold 272 in which the pumps 280, 282, valve 284, and pressure sensor 286 are mounted into the manifold 272 on a plane for slim profile within the second internal compartment 254. The manifold 272 can include chambers 288 formed into the second major surface in which the chambers are fluidically coupled to the single passageway within the manifold 272. The chambers are configured to receive the pumps 280, 282, and valve 284 and the pressure sensor 286. The manifold 272 can receive a piezoelectric pump. The manifold 272 can receive a cover 290 over the fluidic components 274, which can be hermetically sealed to the second major surface.

The example actuation device 208 can include kink resistant tubing 292 that can extend through the header 226 and attached to the ports 276, 278 via components such as a barb 294 and O-rings. The kink resistant tubing 292 can be attached to the tubing 210, 212 to fluidically couple the actuation device 208 to the reservoir 204 and the inflatable member, such as the cylinders 202.

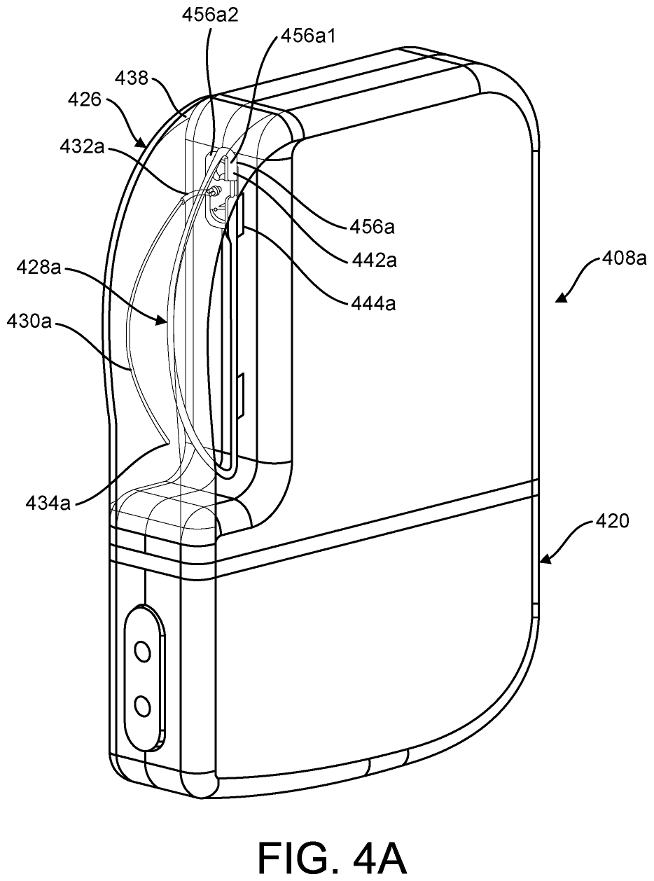

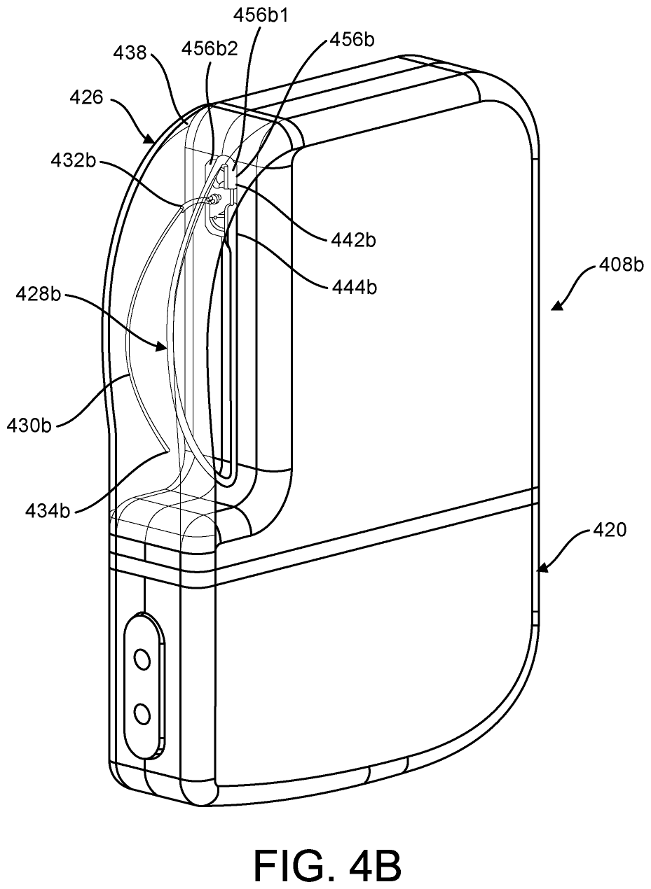

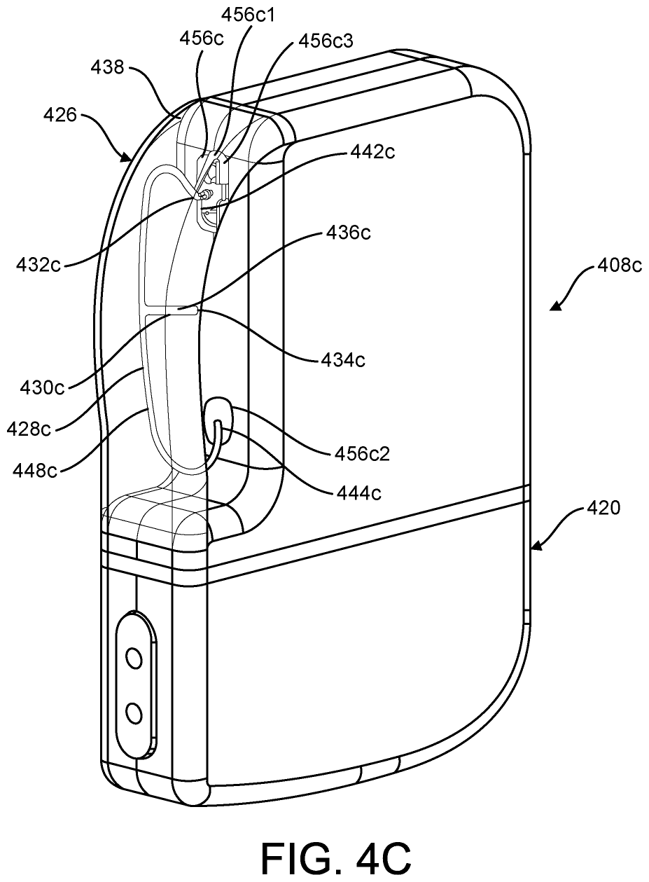

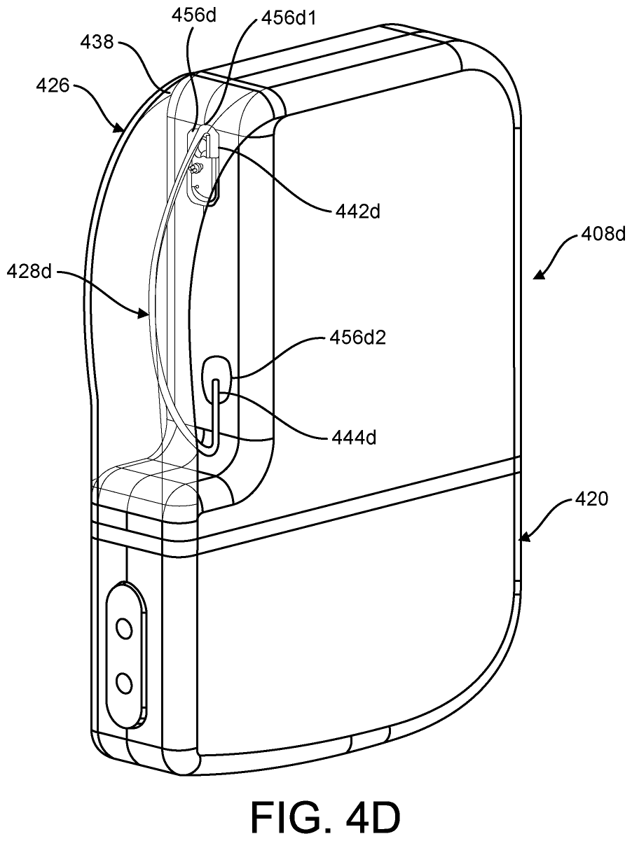

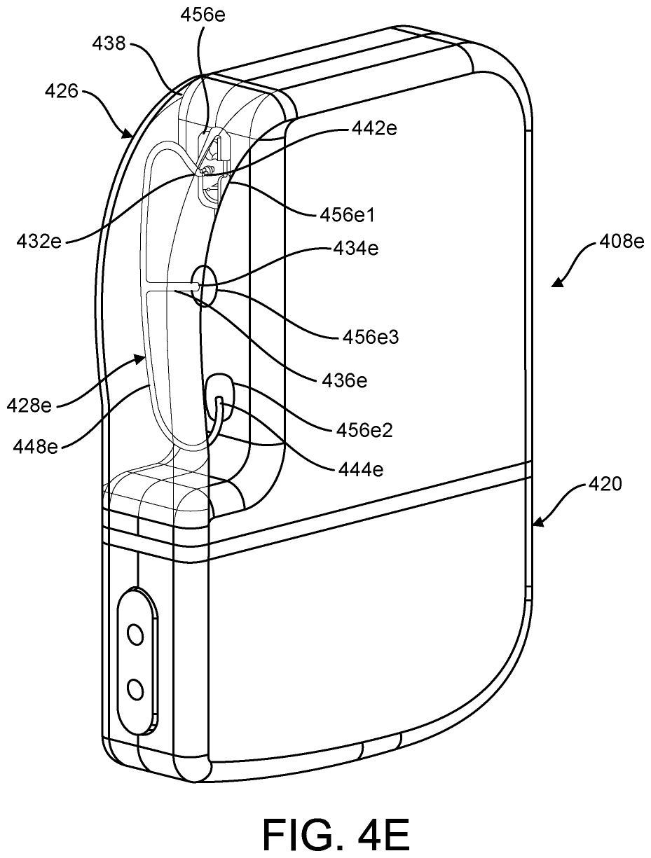

FIG. 4A illustrates another embodiment of an actuation device 408a, which may be incorporated into the implantable medical device 200 of FIG. 2 instead of actuation device 208. For instance, the alternate implantable medical device can include an inflatable member, such as a pair of inflatable cylinders 202, a reservoir 204 that may be filled with a fluid, such as a sterile saline solution 206, and electromechanical actuation device 408a in a closed system. The reservoir 204 is fluidically coupled to the actuation device 408a via tubing 210, and the actuation device 408a is fluidically coupled to the cylinders 202 via tubing 212. The electromechanical actuation device 408a corresponds with implantable medical device 30 of FIG. 1.

The actuation device 408a includes a hermetically sealed housing 420 formed of a biocompatible material such as titanium or steel. In one example, the housing 420 is formed via a plurality of walls welded together. The actuation device 408a includes an internal fluidics circuit to fluidically couple the reservoir 204 to the cylinders 202 via tubing 210, 212. Internal electronic components in a first internal compartment of the housing 420, powered by a rechargeable power source in the first internal compartment, can provide for the monitoring and control of various operations of the fluidics circuit such as the transfer of fluid 206 between the reservoir 204 and the cylinders 202. The actuation device 408a includes a header 426 to form a second internal compartment 438 that includes power and communication interface structures such as a secondary conductor 428a and an antenna 430a.

The header 426 is configured to allow the transmission of power and communication signals between the antenna 430a and a handset programmer or charger, and between the secondary conductor 428a and the charger. In one embodiment, the header 426 is configured from a dielectric or insulative material, such as a radome constructed from a polymer or an epoxy. In the illustrated embodiment, the header 426 is not hermetically sealed to the housing 420, and components within the second compartment 438 are constructed from a biocompatible material. For instance, the secondary conductor 428a and antenna 430a are constructed from a biocompatible material. In embodiments, the secondary conductor 428a and antenna 430a can be constructed from a stamped titanium support member clad in a gold or silver cover to help carry the current and provide for biocompatibility. In another example, the secondary coil 428a and antenna 430a are constructed from a gold wire. The secondary conductor 428a and antenna 430a are electrically coupled to the electronic component within the housing 420 via a biocompatible hermetic feedthrough 456a. In the illustrated example, the feedthrough 456a includes a first feedthrough connection 456a1 electrically and mechanically coupled to the secondary conductor 428a and to a recharge system of the electronic component within the housing 420 and a separate, second feedthrough connection 456a2 electrically and mechanically coupled to the antenna 430a and to a communication system of the electronic component within the housing 420. In the illustrated embodiment, the secondary conductor 428a and antenna 430a are included as separate members in separate structures. The antenna 430a has an effective length and shape and a first end 432a spaced apart from the second end 434a. The first end 432a of the antenna 430a is coupled to the communication system of the electronic component via the second, or antenna feedthrough connection 456a2, and the second end 434a of the antenna 430a is spaced away from the housing 428 and within the second internal compartment 438. The secondary conductor 428a has an effective length and shape and a first end 442a spaced apart from the second end 444a. In the illustrated embodiment, the first end 442a of the secondary conductor 428a is coupled to the recharge system of the electronic component via the first, or secondary conductor feedthrough connection 456a1, and the second end 444a of the secondary conductor 428a is electrically coupled to the housing 420, which is a conductive housing in the illustrated embodiment. In this respect, the conductive housing 420, such as a housing made from titanium, is included as part of the secondary conductor system to receive power signals. Without being bound to a particular theory, the second end 444a of the secondary conductor 428a conductively attached, such as welded, to the housing 420 captures eddy currents as a result of the wireless power transfer.

FIG. 4B illustrates another configuration of a secondary conductor 428b and antenna 430b in an embodiment of actuation device 408b, which may be incorporated into the implantable medical device 200 of FIG. 2 instead of actuation device 208. The actuation device 408b includes a hermetically scaled housing 420 formed of a biocompatible material such as titanium or steel forming a first internal compartment that includes a recharge system and a communication system of an electronic component. The actuation device 408b includes a header 426 to form a second internal compartment 438 that includes power and communication interface structures such as a secondary conductor 428b and an antenna 430b. The header 426 is configured from a dielectric or insulative material, such as a radome constructed from a polymer or an epoxy. In the illustrated embodiment, the header 426 is not hermetically sealed to the housing 420, and components within the second compartment 438 are constructed from a biocompatible material. For instance, the secondary conductor 428b and antenna 430b are constructed from a biocompatible material. The secondary conductor 428b and antenna 430b are electrically coupled to the electronic component within the housing 420 via a biocompatible hermetic feedthrough connection 456b. In the illustrated example, the feedthrough connection 456b includes two secondary conductor feedthrough connections 456b1, 456b2 electrically and mechanically coupled to the secondary conductor 428b and to a recharge system of the electronic component within the housing 420 and a separate, antenna feedthrough connection 456b3 electrically and mechanically coupled to the antenna 430b and to a communication system of the electronic component within the housing 420. In the illustrated embodiment, the secondary conductor 428b and antenna 430b are included as separate members in separate structures. The antenna 430b has an effective length and shape and a first end 432b spaced apart from the second end 434b. The first end 432b of the antenna 430b is coupled to the communication system of the electronic component via the antenna feedthrough connection 456b3, and the second end 434b of the antenna 430b is spaced away from the housing 428 and within the second internal compartment 438. The secondary conductor 428b has an effective length and rounded shape and a first end 442b spaced apart from the second end 444b. In the illustrated embodiment, the first end 442b of the secondary conductor 428b is coupled to the recharge system of the electronic component via the first secondary conductor feedthrough connection 456b1, and the second end 444b of the secondary conductor 428b is electrically coupled to the recharge system of the electronic component via the second secondary conductor feedthrough connection 456b2. Without being bound to a particular theory, the secondary conductor 428b in the illustrated configuration with both ends 442b and 444b coupled to the recharge system, operates as a single turn coil with less resistance and higher maximum power capability than multiple turn coils. In one example, the power signal can be applied at about 6.78 MHz to reduce eddy currents from the housing, which can limit the charge rate. In the illustrated embodiment, the secondary conductor 438b is not electrically coupled to the housing 420. In this illustrated embodiment, the secondary conductor 438b is not electrically coupled to the housing 420.

FIG. 4C illustrates another configuration of a secondary conductor 428c and antenna 430c in an embodiment of actuation device 408c, which may be incorporated into the implantable medical device 200 of FIG. 2 instead of actuation device 208. The actuation device 408c includes a hermetically scaled housing 420 formed of a biocompatible material such as titanium or steel forming a first internal compartment that includes a recharge system and a communication system of an electronic component. The actuation device 408c includes a header 426 to form a second internal compartment 438 that includes power and communication interface structures such as a secondary conductor 428c and an antenna 430c. The header 426 is configured from a dielectric or insulative material, such as a radome constructed from a polymer or an epoxy. In the illustrated embodiment, the header 426 is not hermetically sealed to the housing 420, and components within the second compartment 438 are constructed from a biocompatible material. The secondary conductor 428c and antenna 430c are electrically coupled to the electronic component within the housing 420 via a biocompatible hermetic feedthrough connection 456c. In the illustrated example, the feedthrough connection 456c includes two secondary conductor feedthrough connections 456c1, 456c2 electrically and mechanically coupled to the secondary conductor 428c and to a recharge system of the electronic component within the housing 420 and a separate, antenna feedthrough connection 456c3 electrically and mechanically coupled to the antenna 430c and to a communication system of the electronic component within the housing 420. In the illustrated embodiment, the secondary conductor 428c and antenna 430c are included as separate members in a common structure at least mechanically coupled together. The antenna 430c has an effective length and shape and a first end 432c spaced apart from the second end 434c. The first end 432c of the antenna 430c is coupled to the communication system of the electronic component via the antenna feedthrough connection 456c3. The second end 434c of the antenna 430c is spaced away from the housing 428 but is terminal to a monopole portion 436c extending from a crossmember 448c of the secondary conductor 428c as illustrated. In some examples, the monopole 436c is electrically coupled to the secondary conductor 428c. For instance, the monopole 436c is mechanically and electrically coupled to the crossmember 448c of the secondary conductor 428c. The secondary conductor 428c has an effective length and shape and a first end 442c spaced apart from the second end 444c.

FIG. 4D illustrates another configuration of a secondary conductor/antenna 428d as a common, conductive structure in an embodiment of actuation device 408d, which may be incorporated into the implantable medical device 200 of FIG. 2 instead of actuation device 208. The actuation device 408d includes a hermetically scaled housing 420 formed of a biocompatible material such as titanium or steel forming a first internal compartment that includes a recharge system and a communication system of an electronic component. The actuation device 408d includes a header 426 to form a second internal compartment 438 that includes power and communication interface structure such as the secondary conductor/antenna 428d. The header 426 is configured from a dielectric or insulative material, such as a radome constructed from a polymer or an epoxy. In the illustrated embodiment, the header 426 is not hermetically sealed to the housing 420, and components within the second compartment 438 are constructed from a biocompatible material. The secondary conductor/antenna 428d is electrically coupled to the electronic component within the housing 420 via a biocompatible hermetic feedthrough connection 456d. In the illustrated example, the feedthrough connection 456d includes two feedthrough connections 456d1, 456d2 electrically and mechanically coupled to the secondary conductor/antenna 428d. A first end 442d of the secondary conductor/antenna 428d is coupled to the first feedthrough connection 456d1, and a second end 444d of the secondary conductor/antenna 428d is coupled to the second feedthrough connection 456d2. The two feedthrough connections 456d1, 456d2 are electrically a coupled to the recharge system of the electronic component. In some embodiments, the feedthrough connection 456d1 and 456d2 are spaced apart. The first feedthrough connection 456d1 is electrically coupled to a communication system of the electronic component. A switch or other connection circuitry can be interposed between the feedthrough connection 456d and the recharge and communication systems to select whether to couple the recharge system to the feedthrough connections 456d1 and 456d2 or the communication system to the feedthrough connection 456d1. In this embodiment, the secondary conductor/antenna 428d has an effective length and shape configured to receive power signals and to receive communication signals. The switch or connection circuitry can be used to select the functionality of the secondary conductor/antenna 428d by connecting to either the recharge system, in which case the secondary conductor/antenna 428d operates to receive power signals, or to the communication system, in which case the secondary conductor/antenna 428d operates to receive communication signals.

FIG. 4E illustrates another configuration of a secondary conductor/antenna 428e as a common, conductive structure in an embodiment of actuation device 408e, which may be incorporated into the implantable medical device 200 of FIG. 2 instead of actuation device 208. The actuation device 408e includes a hermetically scaled housing 420 formed of a biocompatible material such as titanium or steel forming a first internal compartment that includes a recharge system and a communication system of an electronic component. The actuation device 408e includes a header 426 to form a second internal compartment 438 that includes power and communication interface structure such as the secondary conductor/antenna 428e. The header 426 is configured from a dielectric or insulative material, such as a radome constructed from a polymer or an epoxy. In the illustrated embodiment, the header 426 is not hermetically scaled to the housing 420, and components within the second compartment 438 are constructed from a biocompatible material. The secondary conductor/antenna 428e is electrically coupled to the electronic component within the housing 420 via a biocompatible hermetic feedthrough connection 456e. In the illustrated example, the feedthrough connection 456e includes two feedthrough connections 456e1, 456e2 electrically and mechanically coupled to the secondary conductor/antenna 428e. A first end 442e of the secondary conductor/antenna 428e is coupled to the first feedthrough connection 456e1, and a second end 444e of the secondary conductor/antenna 428e is coupled to the second feedthrough connection 456e2. The secondary conductor/antenna 428e also includes a conductive monopole stub 436e extending from between the first and second ends 442e, 444e of a conductive crossmember 448e. The monopolar stub 436e is mechanically and electrically coupled to the crossmember 448e. The monopole stub 436e includes a monopole end 446e, which is coupled to a third feedthrough connection 456e3 of feedthrough connection. The first and second feedthrough connections 456e1, 456e2 are electrically a coupled to the recharge system of the electronic component. In some embodiments, the feedthrough connection 456e1 and 456e2 are spaced apart. The first and third feedthrough connections 456e1, 456e3 are electrically coupled to a communication system of the electronic component. A switch or other connection circuitry can be interposed between the feedthrough connection 456e and the recharge and communication systems to select whether to couple the recharge system to the first and second feedthrough connections 456e1 and 456e2 or the communication system to the first and third feedthrough connections 456e1, 456e3. In this embodiment, the secondary conductor/antenna 428e has an effective length and shape configured to receive power signals and, along with the monopole stub 436e to receive communication signals. The switch or connection circuitry can be used to select the functionality of the secondary conductor/antenna 428e by connecting to either the recharge system, in which case the secondary conductor/antenna 428e operates to receive power signals, or to the communication system, in which case the secondary conductor/antenna 428e operates to receive communication signals.

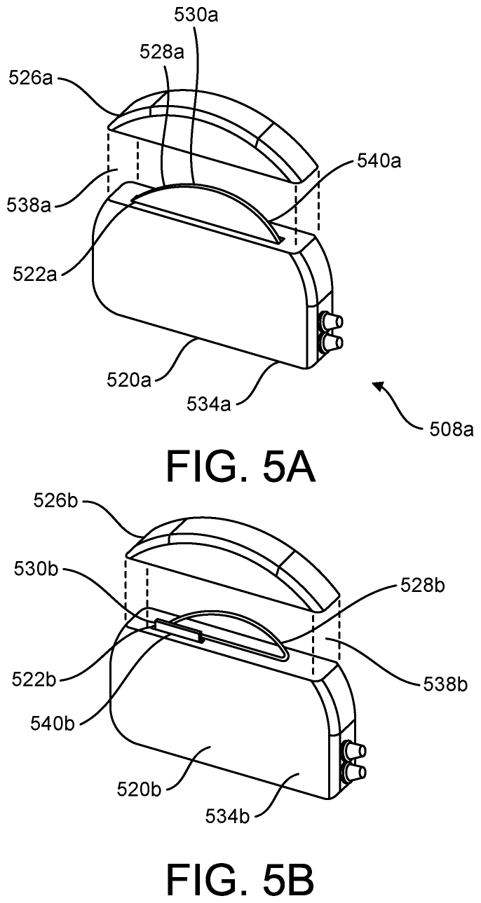

FIG. 5A illustrates another embodiment of an actuation device 508a, which may be incorporated into the implantable medical device 200 of FIG. 2 instead of actuation device 208. For instance, the alternate implantable medical device can include an inflatable member, such as a pair of inflatable cylinders 202, a reservoir 204 that may be filled with a fluid, such as a sterile saline solution 206, and electromechanical actuation device 508a in a closed system. The reservoir 204 is fluidically coupled to the actuation device 508a via tubing 210, and the actuation device 508a is fluidically coupled to the cylinders 202 via tubing 212. The electromechanical actuation device 508a corresponds with implantable medical device 30 of FIG. 1.

The actuation device 508a includes a sealed housing 520a formed of a biocompatible material such as titanium or steel. In one example, the housing 520a is formed via a plurality of walls welded together to form a hermetic seal. The actuation device 508a includes an internal fluidics circuit to fluidically couple the reservoir 204 to the cylinders 202 via tubing 210, 212. Internal electronic components in a first internal compartment 534a of the housing 520a, powered by a rechargeable power source in the first internal compartment, can provide for the monitoring and control of various operations of the fluidics circuit such as the transfer of fluid 206 between the reservoir 204 and the cylinders 202. The actuation device 508a includes a hermetically scaled header 526a that forms a second internal compartment 538a that includes power and communication interface structures such as a secondary conductor 528a and an antenna 530a. In the illustrated embodiment, the housing 520a is hermetically sealed to the header 526a.

The header 526a is configured to allow the transmission of power and communication signals between the antenna 530a and a handset programmer or charger, and between the secondary conductor 528a and the charger. In one embodiment, the header 526a is configured from a nonconductive, bioceramic material such as one including zirconia 3Y-TZP. The header 526a can include a metal flange attached to the ceramic material via a ceramic-metal braise. Located within the hermitically sealed second internal compartment 538a capable of signal transmission, the secondary conductor 528a and antenna 530a can be constructed from a low resistance material such as copper, which tends to generate less heat during signal transmission than higher resistance materials. In the illustrated embodiment, the housing 520a includes an opening 522a so the first internal compartment 534a is in communication with the second internal compartment 538a. The secondary conductor 528a can be implemented as a coil disposed on a circuit board 540a with the antenna 530a extending from the first internal compartment 534a into the second internal compartment 538a so as to receive signals through the header 526a and also couple to the electronic component, which can be disposed on circuit boards such as illustrated in FIG. 3.

FIG. 5B illustrates another embodiment of an actuation device 508b, which may be incorporated into the implantable medical device 200 of FIG. 2 instead of actuation device 208. For instance, the alternate implantable medical device can include an inflatable member, such as a pair of inflatable cylinders 202, a reservoir 204 that may be filled with a fluid, such as a sterile saline solution 206, and electromechanical actuation device 508b in a closed system. The reservoir 204 is fluidically coupled to the actuation device 508b via tubing 210, and the actuation device 508b is fluidically coupled to the cylinders 202 via tubing 212. The electromechanical actuation device 508a corresponds with implantable medical device 30 of FIG. 1.

The actuation device 508b includes a scaled housing 520b formed of a biocompatible material such as titanium or steel. In one example, the housing 520b is formed via a plurality of walls welded together to form a hermetic seal. The actuation device 508b includes an internal fluidics circuit to fluidically couple the reservoir 204 to the cylinders 202 via tubing 210, 212. Internal electronic components in a first internal compartment 534b of the housing 520b, powered by a rechargeable power source in the first internal compartment, can provide for the monitoring and control of various operations of the fluidics circuit such as the transfer of fluid 206 between the reservoir 204 and the cylinders 202. The actuation device 508b includes a hermetically sealed header 526b that forms a second internal compartment 538b that includes power and communication interface structures such as a secondary conductor 528b and an antenna 530b. In the illustrated embodiment, the housing 520a is hermetically sealed to the header 526b such as in the manner and construction of the actuation device 508 of FIG. 5A. In the illustrated embodiment, the housing 520b includes an opening 522b so the first internal compartment 534b is in communication with the second internal compartment 538b. The secondary conductor 528b and antenna 530b can be implemented as conductive structures as illustrated in FIGS. 4A-4E, or other conductive structures coupled to a circuit board 540b, such as a printed circuit board assembly, extending from the first internal compartment 534b into the second internal compartment 538b so as to receive signals through the header 526b and also couple to the electronic component, which can be disposed on circuit boards such as illustrated in FIG. 3.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An implantable urological device, comprising:
a housing forming a first internal compartment;
an electronic component and a rechargeable power source disposed within the first internal compartment, the electronic component including a treatment system, a communication system, and a recharge system, the recharge system coupled to the rechargeable power source;
a header coupled to the housing, the header forming a second internal compartment;
a secondary conductor disposed within the second internal compartment and electrically coupled to the electronic component, the secondary conductor configured to receive a wireless power signal, wherein the recharge system is configured to charge the rechargeable power source from the power signal received from the secondary conductor; and
an antenna disposed within the second internal compartment and electrically coupled to the electronic component, the antenna configured to a receive a wireless communication signal, wherein the communication signal is provided to the communication system.

2. The implantable urological device of claim 1, wherein the power source is a rechargeable power source including a rechargeable battery.

3. The implantable urological device of claim 2, wherein the implantable urological device is an inflatable penile prosthesis.

4. The implantable urological device of claim 3, and further comprising a fluidics circuit disposed within the housing and electrically coupled to the treatment system.

5. The implantable urological device of claim 4, wherein housing includes a third internal compartment, the fluidics circuit disposed within the third internal compartment, and the third internal compartment hermetically sealed from the first and second internal compartments.

6. The implantable urological device of claim 5, wherein the fluidics circuit includes a pump assembly.

7. The implantable urological device of claim 5, wherein the fluidics circuit includes a manifold hermetically sealed to the housing to form the third internal compartment.

8. The implantable urological device of claim 1, wherein the first internal compartment is hermetically sealed from the second internal compartment, and the secondary conductor and the antenna are electrically coupled to the electronic component via a hermetic feedthrough system.

9. The implantable urological device of claim 8, wherein the hermetic feedthrough system is a common feedthrough electrically and mechanically coupling the secondary conductor and the antenna to the electronic component via a switch configured to select one of the power signal and the communication signal.

10. The implantable urological device of claim 8, wherein the hermetic feedthrough system includes a secondary coil feedthrough electrically and mechanically coupling the secondary conductor to a recharge system of the electronic component and wherein the hermetic feedthrough system includes an antenna feedthrough electrically and mechanically coupling the antenna to a communication system of the electronic component.

11. The implantable urological device of claim 8, wherein the antenna is a monopole stub mechanically coupled to the secondary conductor.

12. The implantable urological device of claim 8, wherein the housing is electrically conductive, and the secondary conductor includes a first end electrically coupled to the feedthrough system and a second end electrically coupled to the housing.

13. The implantable urological device of claim 8, wherein the housing is electrically conductive, and the secondary conductor is electrically insulated from the housing.

14. The implantable urological device of claim 1, wherein the first and second internal compartments are hermetically sealed within the housing and header, and at least one of the of the secondary conductor and the antenna include copper.

15. An implantable urological device, comprising:
a housing forming a first internal compartment;
an electronic component disposed within the first internal compartment;
a header coupled to the housing, the header forming a second internal compartment;
a secondary conductor disposed within the second internal compartment and electrically coupled to the electronic component, the secondary conductor configured to receive a wireless power signal; and
an antenna disposed within the second internal compartment and electrically coupled to the electronic component, the antenna configured to a receive a wireless communication signal.

16. The implantable urological device of claim 15, wherein the implantable urological device is included in a medical system further comprising a remote charger and a remote programmer wherein the remote charger is wirelessly coupleable to the secondary coil to provide the transcutaneous power transfer, and wherein the programmer is in radiofrequency communication with the electronic component via the antenna.

17. The implantable urological device of claim 16, wherein the programmer includes a software application operating on a mobile computing device to activate the implantable medical device.

18. An implantable urological device, comprising:
a reservoir configured to receive a fluid;
an inflatable member in fluid communication with the reservoir; and
an implantable medical actuation device, comprising:
a housing forming a first internal compartment;
an electronic component and a rechargeable power source disposed within the first internal compartment, the electronic component including a treatment system, a communication system, and a recharge system, the recharge system coupled to the rechargeable power source;
a header coupled to the housing, the header forming a second internal compartment;
a secondary conductor disposed within the second internal compartment and electrically coupled to the electronic component, the secondary conductor configured to receive a wireless power signal, wherein the recharge system is configured to charge the rechargeable power source from the power signal received from the secondary conductor; and
an antenna disposed within the second internal compartment and electrically coupled to the electronic component, the antenna configured to a receive a wireless communication signal, wherein the communication signal is provided to the communication system.

19. The implantable urological device of claim 18, wherein the inflatable member includes a plurality of inflatable cylinders in fluid communication with the medical actuation device and the reservoir, the plurality of inflatable cylinders configured to be disposed within a corpora cavernosa of a penis.

20. The implantable urological device of claim 18, wherein the medical instrument is configured to be disposed within a retropubic space.
